(19) United States
(12) Patent Application Publication
(10) Pub. No.: US 2015/0165231 A1
(43) Pub. Date: Jun. 18, 2015

(54) PHOTOTHERAPY GEL PACK

(71) Applicant: KONINKLIJKE PHILIPS N.V., EINDHOVEN (NL)

(72) Inventors: Michael Martin Scheja, Eindhoven (NL); Xiao Xin Wang, Eindhoven (NL); Jing Bo Ma, Eindhoven (NL)

(21) Appl. No.: 14/408,619

(22) PCT Filed: Jun. 18, 2013
(86) PCT No.: PCT/IB2013/055003

§ 371 Date: Dec. 17, 2014

Related U.S. Application Data
(60) Provisional application No. 61/662,441, filed on Jun. 21, 2012.

(51) Int. Cl. A61N 5/06; A61F 7/08; A61M 37/00

(52) U.S. Cl. CPC A61N 5/0625; A61M 37/00; A61N 5/062; A61F 7/08; A61M 2037/0007

(57) ABSTRACT

A hot/cold therapy and light therapy (phototherapy) provide synergistic effects for a user. An improved gel pack is provided wherein a flexible circuit with at least one light source is provided. The at least one light source emits light through the gel and onto the target treatment area. The flexible circuit is sealed and embedded in the gel pack to allow the gel pack (including the flexible circuit therein) to be washed and cleaned easily. Furthermore, the improved gel pack may be assembled in a layered structure to provide the waterproof gel pack, e.g., sealing the gel in the gel pack and embedding a flexible circuit in the gel pack. To improve durability, the flexible circuit may be supported by a layer which forms the outer covering of the gel pack.

### FIELD OF THE INVENTION

The present disclosure relates to phototherapy assemblies comprising flexible, light emitting diodes (LEDs) embedded in gel, in particular, human wearable phototherapy gel packs with heating and/or cooling functions.

### BACKGROUND OF THE INVENTION

Light therapy, such as applying light of relatively low energy density onto a living human or animal, may be used to modulate and/or affect cell activity. This type of light therapy, also referred to as photobiomodulation, is characterized by the application of light without causing substantial thermal effects. Photobiomodulation is known to have, e.g., cosmetic and/or therapeutic benefits for tissues like skin, muscles, etc.

Examples of light sources used in light therapy may include lasers and light emitting diodes (LEDs). LEDs, in particular, are preferred in certain applications for having the ability to illuminate a larger area than a laser. Light emitted from LEDs may decrease wrinkles and skin roughness by increasing collagen and elastin synthesis, and reduce pigmentation in human skin. Furthermore, the emitted light may protect against subsequent photo damage, prevent post-inflammatory hyperpigmentation and reduce scar formation during healing. Moreover, the illumination from blue or infrared LEDs may cause generation and release of nitric oxide, which may subsequently lead to pain relief.

Gel-based ice and heat packs are widely used for first aid and therapy to treat aches, bruises, pains, sprains, and strains. Cold packs may be used to reduce swelling or to help recover from the sun. The gel inside the gel packs is provided to store cold/warmth such that a target area can be slowly cooled or heated during therapy. Typically, the gel packs are provided with a flexible package material such that the pack may be formed and applied to uneven target treatment areas such as limbs, faces, joints, etc.

US 2006/0235494 A1 describes a therapeutic device including a container for applying at least one of hot therapy and cold therapy. The container, having a non-electrical agent for applying at least one of hot therapy and cold therapy, has a pocket on an outer surface of the container. A member having at least one light source for emitting therapeutic light is sized to be removably positioned within the pocket.

### SUMMARY OF THE INVENTION

Therapeutic device disclosed in the prior art has several disadvantages. The removable member having the light source is placed in a pocket outside the container (i.e., the gel pack). The removable member must be inserted into the pocket before using the device, and also, the member must be removed out of the pocket if the user wishes to heat the gel pack with hot water. Additional steps for assembling and disassembling the therapeutic device may cause user confusion or ineffective usage of the device due to erroneous operator error. For instance, a user may cause the device to malfunction by submerging the gel pack and the removable member (not waterproof) together in hot water to heat up the gel pack. In another instance, the user may insert the removable member where the light is being applied in the wrong direction, away from the target treatment area. In yet another instance, the removable member may fall off and/or become lost, leading to a non-functioning therapeutic device. The gel in the prior art is provided farther away from the target treatment area than the light source, leaving the light source close to the target treatment area. If the light source emits heat, the heat emitted may cause damage to the target treatment area. Furthermore, this configuration delivers the thermal effects from a hot/cold gel inefficiently because the heat or coldness must go through the light source to reach the target treatment area.

Accordingly, improved therapeutic apparatuses as disclosed aims to alleviate at least one of the problems describe above.

A hot and/or cold gel pack may be applied to treat a target treatment area of a living being, such as an adult, a child, a baby, an animal, etc. A substantially flexible circuit with at least one light source is embedded in the gel of the gel pack. Light from the at least one light source may emit through the gel and onto the target treatment area. Accordingly, the phototherapy gel pack provides both hot/cold therapy as well as light therapy. The gel pack is advantageously waterproof. In one embodiment, layers of the gel pack are sealed to form a waterproof layered structure, wherein a gel is held in the gel pack and a flexible circuit having at least one light source is embedded in the gel pack.

The gel pack may be advantageously flexible to enable a user to apply the gel pack as closely and fitted as possible onto the anatomy of the target treatment area. The flexibility of the phototherapy gel pack may allow even, homogeneous and/or effective distribution of illumination to the target treatment area, even when the anatomy is “hilly” or “bumpy” (uneven or not smooth). In some embodiments, at least the outer surface material may be flexible and may enable a user to apply the gel pack and/or deform the gel pack to fit over the target treatment area. In some embodiments, the outer surface material is stretchable to provide a better fit over the target treatment area. Stretchability or elasticity may also allow the gel pack to be more durable and usable, since some treatment applications may require a relatively tight fit to the treatment area, which may require relatively more deformation of the gel pack.

The phototherapy gel pack may have an outer surface material that is waterproof, both to keep the gel inside the gel pack from leaking, as well as to prevent water or moisture from entering the gel pack, e.g., damaging the flexible circuit and/or the at least one light sources. The outer surface material is advantageously easy to clean and/or sanitize. For instance, a user may wipe the outer surface with rubbing alcohol or soap solution to sanitize the gel pack for further reuse/storage. In some embodiments, the outer surface material is a smooth material to avoid collecting contamination/dirt and facilitate cleaning.

In one embodiment, a phototherapy assembly comprises at least one light source for providing a therapeutic effect to a target treatment area of a living being. The assembly includes first waterproof and flexible layer applicable onto the target treatment area. The waterproof first layer advantageously provides an occlusion effect on the target treatment area, which may promote absorption of topical treatments that may be provided between the first layer and the target treatment area.

The assembly further comprises a second waterproof and flexible layer, which advantageously is sealed with the first layer to provide a waterproof assembly. The assembly further comprises a gel in between the first layer and the second layer. The gel, depending on the therapeutic, medical and/or cosmetic application, may be used to provide any of its variety of effects. For instance, the gel may provide heat or cold treatment during phototherapy treatment for synergistic effects. The gel may be used, in some instances, for thermal management.

The assembly further comprises a flexible circuit in between the first layer and the second layer, said flexible circuit having at least one light source for emitting light. The first layer and the second layer are sealed to provide a waterproof gel pack having the flexible circuit embedded in the waterproof gel pack. The waterproof gel pack assembly is advantageously able to be submerged in warm or hot water to heat the gel pack, and/or facilitate cleaning of the gel pack to promote reuse. The at least one light source emits light through the gel and the first layer and onto the target treatment area. The placement of the gel between the light source and the target treatment area allows the gel to be used as, e.g., a spacer or buffer. Furthermore, the thermal treatment provided by the gel is more effective if placed closer to the target treatment area.

In some embodiments, the second layer (directly) supports the flexible circuit. In some other embodiments, a gel may be provided between the second layer and the flexible circuit for, e.g., added thermal capacity.

In some embodiments, the waterproof gel pack having a flexible circuit embedded therein is constructed of layers that are sealed together, wherein one of the layers supports said flexible circuit. For instance, the first layer, the gel, the second layer, and the flexible circuit are sealed together in a layered structure. The layered structure provides more structural support of the gel pack. A layered structure may be easier to manufacture. The layered structure may provide better support to, e.g., tearable flexible circuit, than a gel pack where the flexible circuit may be free flowing in the gel.

In one embodiment, the assembly may comprise a sealing material between the flexible circuit and the gel. Said sealing material seals the gel with the first layer. The sealing material also seals the flexible circuit with the first layer and the gel. The sealing material provides adhesion between at least parts of the first layer and at least parts the flexible circuit. The seal provided by the sealing material is advantageously durable to prevent the gel from leaking out of the assembly. The sealing material may advantageously protect the flexible circuit from the gel and/or moisture/water outside the assembly.

In one embodiment, the assembly comprises a seal that seals the seams of the first layer and the second layer to provide the waterproof gel pack. A seal as such may be provided in addition or alternative to a sealing material. The seal advantageously prevents the gel from leaking out of the waterproof assembly (likewise to provide water protection for the parts held in the assembly (e.g., the flexible circuit).

In one embodiment, the assembly further comprises said second waterproof and flexible layer comprises a first water protective film deposited on a first side of the flexible circuit. The first water protective film provides protection of the flexible circuit from any damage that could be caused by the gel and/or moisture/water outside the assembly. The water protective film enables easier cleaning. In some embodiments, the water protective film is provided as an outer layer of the assembly, which may be applied to the target treatment area or not points away from the target treatment area.

In one embodiment, the assembly further comprises a second water protective film deposited on a second side of the flexible circuit, wherein the second side of the flexible circuit has the at least one light source. The second water protective film provides protection of the flexible circuit from any damage that could be caused by the gel and/or moisture/water outside the assembly. In some embodiments, the second water protective film acts as a sealing material for sealing the gel and the flexible circuit together.

In one embodiment, the flexible circuit further comprises at least one electrical heating part and/or the at least one light source of the flexible circuit comprises at least one heat producing light source. Heat may be produced by the electrical heating part and/or the light source during treatment, advantageously to provide the synergistic effects of thermal and photo therapy.

In one embodiment, the gel is held by the first layer in a plurality of compartments to provide substantially even distribution of the gel over the flexible circuit. The even distribution of gel allows a more even illumination of the target treatment area, and/or provides a more even distance between the at least one light source and the target treatment area. In some embodiments, a minimum distance may be maintained between the at least one light source and the target treatment area to avoid harm or burning of the target treatment area if the at least one light source is heated and/or at least one electrical heating part is provided.

In some embodiments, the gel may be used as thermal management. For instance, if at least one light source includes at least one high-powered and/or heat producing light source such as high-powered light emitting diodes, the gel may be advantageously provided to absorb/buffer some of the thermal energy provided by the at least one light source and/or an electrical heating part. The thermal energy, without thermal management, can become too hot for the target treatment area. But with the gel as thermal management, the thermal energy from the at least one light source and/or the electrical heating part may then be provided to the target treatment area as beneficial heat therapy, e.g., by applying heat to the target treatment area slowly over a period of time. When light therapy and heat therapy is combined, the synergistic effects as disclosed herein, such as increased uptake of topical formula at the target treatment area, are achieved.

In one embodiment, the assembly comprises an internal power source embedded in the gel pack, and electrically coupled to the flexible circuit, and/or a waterproof connector and an external power source connectable to the waterproof connector to power the flexible circuit. Power source(s) enable the flexible circuit within a waterproof gel pack to have power during therapy.

In one embodiment, a topical formula deposited on the first layer of the assembly, wherein said topical formula is applicable to the target treatment area. The topical formula may be thermally/optically activated, and/or whose absorption is enhanced by the occlusion effect of a waterproof layer applied to the target treatment area.

In one embodiment, a phototherapy system comprises any one of the phototherapy assemblies described herein, and a docking station. The docking station may include at least one heating and/or cooling element for heating the gel of the gel pack, and/or a power source for charging at least one power storage part of the gel pack. Advantageously, the docking station provides a way to heat or cool the gel pack and/or provide the power needed by the flexible circuit during therapy.

In some embodiments, a method of phototherapy includes applying a phototherapy gel pack onto a target treatment area of a living being, wherein the gel pack may be used as a hot pack or a cold pack. Further, the phototherapy method may include providing a topical formula between the phototherapy gel pack and the target treatment area.

The topical formula is thermally and/or optically activated. In some embodiments, for photodynamic therapy, the topical formula includes a photosensitizer. Generally, heat treatment and/or occlusion of the skin may enhance absorption of topical formulas into the skin of the target treatment area. In some embodiments, the topical formula is advantageously enhanced if certain thermal or optical conditions are met (e.g., heat activated).

Embodiments and their advantages described in the summary is not meant to be limiting, but rather is meant as illustrative embodiments for the disclosure.

### BRIEF DESCRIPTION OF THE DRAWINGS

In the drawings:

FIG. 1 depicts an illustrative wearable phototherapy gel pack, according to one embodiment of the disclosure;

FIG. 2 depicts a top view and a side view of an illustrative flexible circuit (cross section) embeddable in a gel pack, according to one embodiment of the disclosure;

FIG. 3a depicts a schematic of an exemplary phototherapy gel pack, according to one embodiment of the disclosure;

FIG. 3b depicts a schematic of another exemplary phototherapy gel pack, according to one embodiment of the disclosure;

FIG. 3c depicts a schematic of another exemplary phototherapy gel pack, according to one embodiment of the disclosure;

FIG. 4a depicts a schematic of an exemplary phototherapy gel pack connectable to a power source, according to one embodiment of the disclosure;

FIG. 4b depicts a schematic of another exemplary phototherapy gel pack having an internal power source, according to one embodiment of the disclosure;

FIG. 5a depicts a schematic of an exemplary phototherapy gel pack connectable to a station, according to one embodiment of the disclosure; and

FIG. 5b depicts a schematic of another exemplary phototherapy gel pack having an internal power source, said gel pack connectable to a station, according to one embodiment of the disclosure.

### DETAILED DESCRIPTION OF EMBODIMENTS

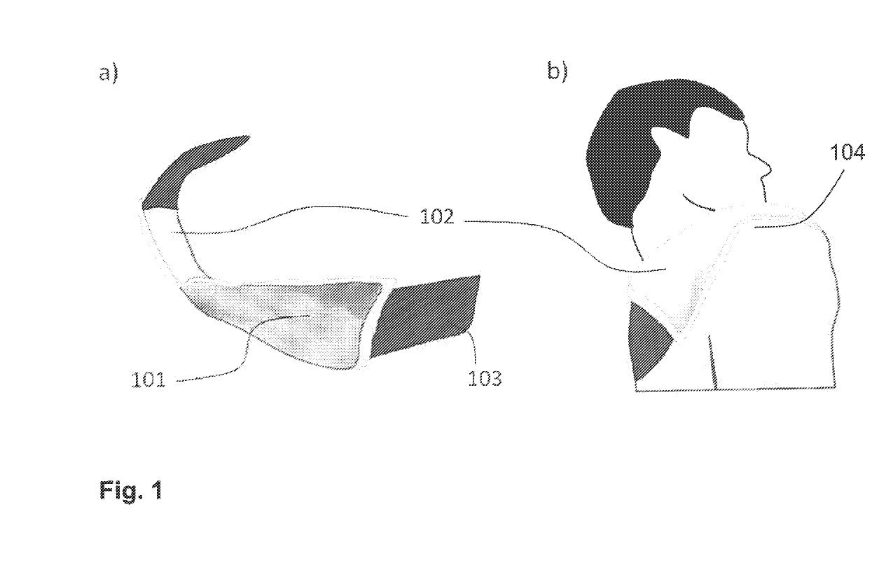

FIG. 1 depicts an illustrative wearable phototherapy gel pack, according to one embodiment of the disclosure. The gel pack may include at least two sheets or layers, and said sheets/layers form a layered structure of a gel pack. In some embodiments, the seams of the layers are sealed to prevent the gel and/or other parts therein from leaking. In certain embodiments, the layers are sealed by, e.g., adhering the layers and a flexible circuit having at least one light source together to form a waterproof, layered structure of the gel pack.

The layers may be sealed using heat, chemical, adhesive, mechanical and/or any other suitable methods and/or means. For instance, at least part of the surfaces of the layers may be bonded or sealed together to form a sealed, waterproof pack to hold the gel and the flexible circuit. As such, said flexible circuit having the at least one light source is advantageously embedded in the gel pack.

In this exemplary embodiment, the gel pack has at least two layers, a first layer 101 and a second layer 102, which form a covering or outer surface of the gel pack. The two layers are sealed to form a waterproof gel pack. In certain embodiments, the layers may be sealed to each other at the edges of the layers to ensure that a gel inside the gel pack is held without leakage, and a flexible circuit having at least one light source is protected from water or moisture outside of the gel pack. In some embodiments, the layers may be sealed to form the waterproof gel pack by adhering the layers together, including embedding a gel and a flexible circuit between the layers. In some embodiments, the light source may produce heat, and/or the flexible circuit may include an electrical heating part.

A first layer 101 may be applied towards the skin or tissue of a living being. The first layer is advantageously applicable to a target treatment area 104. The target treatment area may include any (external) parts of a human body, such as scalp, forehead, face, neck, back, chest, stomach, groin, buttocks, legs, feet, etc. Ointments, creams, wax, powder, gels, fluids, and/or liquids may be applied between the first layer and the target treatment area. The applied substances may be medicated. Phototherapy using these applied substances may be suitable for therapeutic, medical and/or cosmetic use.

The applied substance (e.g., topical treatment/formula), may be applied onto the target treatment area before treatment, and/or applied onto the first layer 101 before treatment. In some embodiments, the applied substances may be (pre-deposited onto and) sealed on the first layer with a removable (and waterproof) seal, such that the seal may be removed before use, and the applied substance is exposed to the target treatment area during use. The seal may be used again to seal any remaining substance back onto the first layer. Then the gel pack maybe advantageously cooled/heated again for later use.

The first layer advantageously comprises a waterproof and/or water impermeable material, which provides occlusion of the skin when applied onto the target treatment area. In some embodiments, occlusion, optionally in combination with heat released from the gel pack, increases the absorption of topical formulas and/or treatments provided between the gel pack and the target treatment area. In certain embodiments, the topical formula and/or treatment is thermally, chemically, and/or optically activated. The topical formula and/or treatment may be for therapeutic and/or cosmetic use.

If the gel pack is used as a heating pack, the heat released from the heat pack to the target treatment area may increase the uptake of applied substances (e.g., topical treatments/formulas) into the skin. The close contact of the gel pack on a target treatment area may also increase the uptake of those applied substances into the skin (e.g., due to occlusion of the skin). Absorption and/or uptake of those substances may result in higher efficiency of the treatment desired for the target treatment area.

If the gel pack is used as a cooling pack, then the cooling effect may work in combination with light therapy to dually provide benefits such as pain relief. In some embodiments, increasing heat and/or temperature of the light sources may decrease the efficiency of the light sources. By providing a cooling pack, the light sources may be cooled to enable higher light intensities, thereby increasing the efficiency of light treatment.

To form a gel pack, the first layer 101 includes a flexible, waterproof outer material that holds a gel (e.g., a gel-like material, a gelatinous substance, a liquid, a thick liquid, etc.). For instance, the first layer may hold a transparent gel, e.g., is 1,2-propylene glycol, water, saline or similar water-salt solution, clear silicone gels, silicone fluids such as polydimethylsiloxane, water/saline with a highly water-binding organic polymer such as hyaluronic acid. Preservatives may be added to lessen microbial degradation. The transparent gel may have a relatively high capacity to store thermal energy such that the first layer is suitable for use as a hot and/or cold gel pack. In some embodiments, the gel may have a high thermal conductivity such as heat transfer fluids or gels, to quickly remove any heat from the at least one light source. Transparent foam may be used in place of the gel, if thermal conductivity is not a factor in the design.

The gel held by the first layer may be substantially transparent or at least translucent as to allow light to pass through. In certain embodiments, the gel may have, e.g., a particular physical and/or chemical structure, such that light may be diffused and/or focused over the target treatment area. In some embodiments, the gel-like material may be provided such that a certain range of frequency of light is allowed to pass through. The gel may be non-toxic, to avoid, e.g., poisoning the user if the gel leaks out of the gel pack.

The gel pack may be provided to the target treatment area at, e.g., room temperature or cooler than room temperature. The gel may be used for thermal management, e.g., using the high capacity of the gel to store thermal energy (or high thermal conductance to quickly remove heat produced by the at least one light source out of the gel pack/towards skin). In some phototherapy treatments, high powered light sources (e.g., high powered LEDs) may be suitable. However, high powered light sources may harm the target treatment area if the heat produced is not managed. The gel provided between the light source and the target treatment area may act as a buffer/spacer, which keeps the heat producing light sources at a safe distance from the target treatment area. The gel may also absorb some of the heat produced by the light sources. The absorbed heat may induce a phase change of the gel material allow the heat applied to the target treatment to be maintained at a particular level (e.g., to avoid over heating of the target treatment area).

The outer surface material may withstand hot temperatures such as hot, boiling, or steaming water (e.g., about 45 degrees Celsius to about 110 degrees Celsius), such that the material will not melt or burn during the heating of the gel pack. The outer surface material may also withstand cold temperatures such as the refrigerator or a freezer (about −20 degrees Celsius to about 15 degrees Celsius), such that the material will not crack and/or become brittle during the cooling of the gel pack.

The first layer 101 may be substantially transparent, such that light may pass through the gel, the first layer and onto the skin of the target treatment area. The first layer may be translucent, such that some of the light emitted from the at least one light source may pass through the layer. The first layer may alternatively be provided with a coating or material that is porous or provided with slits and/or openings such that light may pass through the covering and onto the skin.

In some embodiments, the first layer 101 comprises a washable outer surface to provide easy cleaning of the first layer. The first layer being applied to the skin may become dirty or soiled from sweat, dirt, contaminants, bacteria, creams, ointments/gels, powders, etc. For instance, the first layer may include a smooth surface to enable easier cleaning. A washable surface advantageously provides reusability of the gel pack, e.g., allowing a user to clean the gel pack with a cleansing solution. The first layer may also be flexible, elastic, and/or stretchable to enable the gel pack to fit well over the anatomy of the target treatment area. Because the first layer may be applied to skin, the material may be advantageously non-toxic, hypo-allergenic, and/or comfortable to the touch, even for prolonged use. An anti-bacterial coating may be provided to the first layer to prevent bacterial growth. The outer surface of the first layer may not stick, grab onto, and/or adhere to the skin or hair easily such that the gel pack may be comfortably applied and/or removed from the target treatment area. The first layer 101 may include a topical formula deposited thereon. The topical formula may be deposited manually, or may be sealed onto the first layer using a removable (and waterproof) seal such that it can be exposed to the target treatment area during use.

In some embodiments, the phototherapy gel pack is used in photodynamic therapy. The topical formula may be provided between the gel pack and the target treatment area. The topical formula may include a photosensitizer, whose absorption is facilitated by occlusion and/or heat of the gel pack. With enhanced absorption, the amount of photosensitizer to be provided may be reduced, thereby reducing costs and side effects of photodynamic therapy.

A second layer 102 may point/face away from the target treatment area during use (e.g., not applied to the target treatment area), as seen in part b) of FIG. 1. The second layer may supports a flexible circuit having at least one light source. The flexible circuit may have one side facing towards the gel in the gel pack (gel held by the first layer), such that the light emitted from the at least one light source shines through the gel and onto the target treatment area. The other side facing away from the gel may be adhered or bonded to the second layer. A substance for providing aromatherapy, e.g., thermally activated substance to give off aroma, may be deposited onto the second layer such that an aroma is advantageously given off the second layer to the living being. The substance for providing aromatherapy may be deposited manually, or may be sealed onto the first layer using a removable (and waterproof) seal such that it can be exposed to the target treatment area during use.

The flexible circuit is embedded within layers of the gel pack, e.g., between the first and second layers that are sealed in a waterproof manner. For instance, the flexible circuit is embedded inside the cavity formed between the first and second layers, e.g., in the gel in the gel pack. Said at least one light source configured to emit light through the gel and the first layer, onto the target area of treatment. By providing the gel closer to the target treatment area (as opposed to providing the at least one light sources against the target treatment area), the thermal effects from a hot/cold gel pack is delivered more efficiently and/or directly to the target treatment area.

The second layer may be made of a waterproof and/or water repellent material that protects/seals the flexible circuit from moisture and/or water outside of the gel pack (e.g., the material is waterproof). Together with the first layer, the gel pack formed by the first and second layers is substantially waterproof. The second layer may have an outer material that is easy to clean.

The second layer may be made of a flexible and/or stretchable material, such that flexibility is provided to allow the gel pack to deform, although the flexibility and/or the stretchability of the first layer and the second layer may differ. In some embodiments, the second layer is more flexible/stretchy, since the second layer may be stretched relatively more than the first layer as the gel pack is being wrapped around the target treatment area, the second layer further away from the target treatment area would have a longer arc length.

The second layer, if not intended to be applied to the target treatment area, may be made of material that is opaque. As such, the light emitted from the light source, which may be harmful/undesirable to the eyes or non-target treatment areas can be blocked by an opaque second layer. Alternatively, the second layer maybe translucent, or substantially transparent, advantageously to provide the user with the possibility to view/appreciate the embedded flexible circuit, and/or see that the at least one light source is in operation. In some embodiments, the second layer may be printed with at least one of decals, patterns, instructions for use, temperature indicators, ready to use indicators, warning indicators, etc.

In some embodiments, the first and second layer may both be used for applying onto the target treatment area, thereby providing a double-sided phototherapy gel pack. The flexible circuit may also be double-sided, with at least one light source provided on each side of the circuit. Providing a double-sided gel pack enables the user to use a different side of the gel pack when the cold or warmth of one of the sides of the gel pack had dissipated. The flexible circuit may be configured with a control module electrically coupled to the flexible circuit for switching one side of light source(s) on/off and/or the other side of light source(s) on/off, as to avoid shinning the light for a prolonged period away from the target area, e.g., into the eyes of a user. A user interface component, e.g., a button, a switch, may be provided with the phototherapy gel pack to allow a user to direct the control module to configure the light sources and/or other electrical components (e.g., electrical heating elements, timer, etc.).

The phototherapy gel pack may, in some embodiments, include means and/or at least one part for attaching the gel pack onto the living being such that it may be advantageously used in a hands-free manner. The means or at least one part for attachment provides wearability of the gel pack. The attachment part 103 may enable the gel pack to be held in place on the target treatment area of the living being, even when the living being is moving and/or when the living being is not physically able to hold the gel pack. Such attachment part may include at least one strap attached to or held by the first layer and/or the second layer, such that the straps may be used to attach the gel pack onto the living being. The attachment part may be made of an elastic material (e.g., elastic fabric, latex, etc.) such that it may be fitted snugly onto the living being. The attachment part may be made of a textile material to provide comfortable to the living being. The attachment part may adhere to the living being, e.g., as a removable bandage. The straps may include at least one fastening part, e.g., a hook-and-loop fastener, a buckle, clamp, clip, adhesive, hook or other suitable fastening parts. The attachment part may be adjustable in length to advantageously accommodate living beings of different sizes.

The attachment part, if not waterproof or easily washable, may be at least partly removably attached to the first layer and/or the second layer, as to enable the user to remove (at least part of) the attachment part if the gel pack is to be e.g., cleaned, heated in water, cooled, and/or provided to another user. If the attachment part is washable, removability of the attachment part advantageously enables, e.g., machine or abrasive cleaning/washing of the attachment part. In some embodiments, the attachment part may be removably slidable through a sleeve provided in the second layer. The attachment part may include at least one pocket or sleeve for holding the gel pack, such that the gel pack may be removed from the attachment part when the gel pack is to be washed or heated/cooled. The attachment part may be disposable and/or made of a relatively inexpensive and/or biodegradable material such that the attachment part may be tossed and replaced cost effectively/efficiently.

Although the embodiment shown has a pad-like shape, other types of geometries may be suitable for the gel pack. For instance, the gel pack may have a substantially cylindrical, spherical, or cubed shape. The gel pack may be square, rectangular, circular, triangular, trapezoidal, or of an irregular shape. For instance, the gel pack may be of animal, star, fruit, cartoon shapes for a child-friendly appearance. The gel pack may be shaped to fit over a particular body part, which may be a bumpy area, such as the eyes (e.g., shaped like an eye patch with openings for the eyes to see through the eye patch), the face, the knee, the chest, the shoulders, the hand, etc.

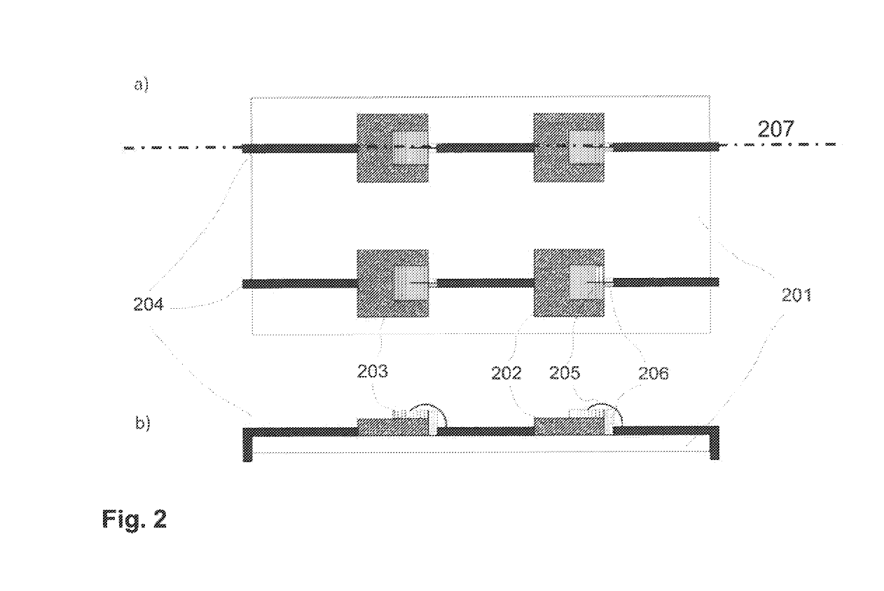

FIG. 2 depicts a top view and a side view of an illustrative flexible circuit (cross section) embeddable in a gel pack, according to one embodiment of the disclosure. The flexible circuit may be adhered/affixed to the second layer inside the gel pack (e.g., within the cavity formed by the first and second layer). In some embodiments, the flexible circuit is within the gel pack and is supported by one of the layers that forms the covering (e.g., the outer surface) of the gel pack. Part a) of the figure shows a top view, and part b) of the figure shows a side view taken at a cross section indicated by dotted line 207. In some embodiments, the thickness of the flexible circuit may range from approximately 0.5 millimeters and 2 millimeters. A flexible circuit that is thin may provide flexibility.

The flexible circuit may include an LED array. The LED array may comprise a plurality of LED elements, arranged in at least one pattern, e.g., web, mesh, grid and/or rows. For illustration, the figure shows four LEDs in the top view for simplicity purposes, but other numbers of LEDs may be used, such as one, a couple, a few, up to thousands of LEDs. An LED may be configured to emit a light in a particular range of frequencies, depending on the therapeutic application. Some of the plurality of LEDs may be configured to emit light in different ranges of frequencies to provide a broader spectrum of light therapy. The flexible circuit has a layer of non-conductive substrate 201, which supports conductive elements thereon that forms the electrically connected array of LEDs. In some embodiments, the non-conductive substrate may be perforated by holes or other cavities, slits, creases, to increase flexibility. One or more LEDs 203 may be mounted on thin (relatively small) plates of a conductive material 202 (or substrate), e.g., copper, silver, etc. Said conductive material may be attached to one or more tracks 204 such that an array of LEDs may be formed. The tracks may include a conductive material, e.g., copper, silver, etc., which connects the LEDs electrically in the array. In particular, a track may connect the thin plates of conductive material via a conductive wire 205 (or line) comprising, e.g., gold. In some embodiments, an insulating material 206 (or substrate) may be provided next to the LED to electrically isolate the track coming from one LED from electrically contacting the thin conductive plate of a following LED to ensure a directed flow of current. Tracks may be fabricated thin enough to provide flexibility of the circuit. The tracks may be electrically connected to a power source.

The construction of thin plates of conductive material connected by thin conductive tracks enables a more flexible design than circuits having many layers of conductive materials. The construction advantageously decreases the amount of conductive and/or metallic substances to be used.

The LED array (or other suitable light sources) may include light sources that are heat-producing, e.g. due to the high power capabilities of the LEDs. The heat-producing LEDs may produce heat that can cause harm, and thus, a gel is provided advantageously between the LEDs and a target treatment area as, e.g., a heat absorbing barrier/buffer.

In some embodiments, the flexible circuit may have LED arrays on more than one side, e.g., with thin plates of conductive material, insulating material, and the conductive wire provided on the other side of the non-conductive substrate. As such, light is advantageously emitted in more than one direction from the flexible circuit.

In some embodiments, the LED array is provided as a thin sheet-like member of the gel pack. In certain embodiments, the LED array may be provided as a ribbon-like member or rope-like member. The LED array may advantageously comprise unwoven or woven rows of ribbon-like members. The thin and flexible, e.g. sheet-like or ribbon-like, characteristic of the LED array may advantageously enable easier manufacturing or transport if the LED array is rollable.

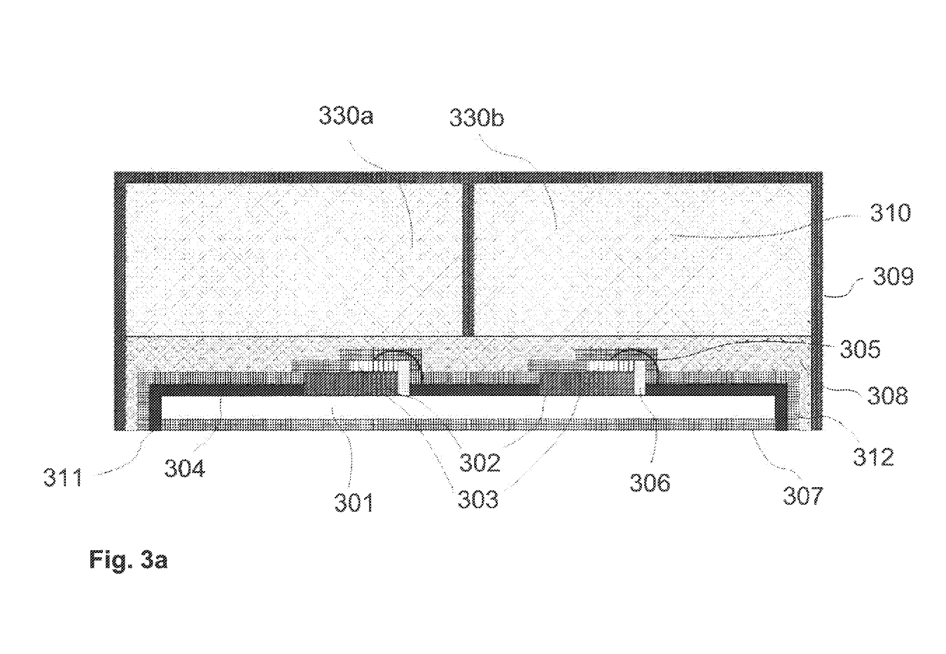

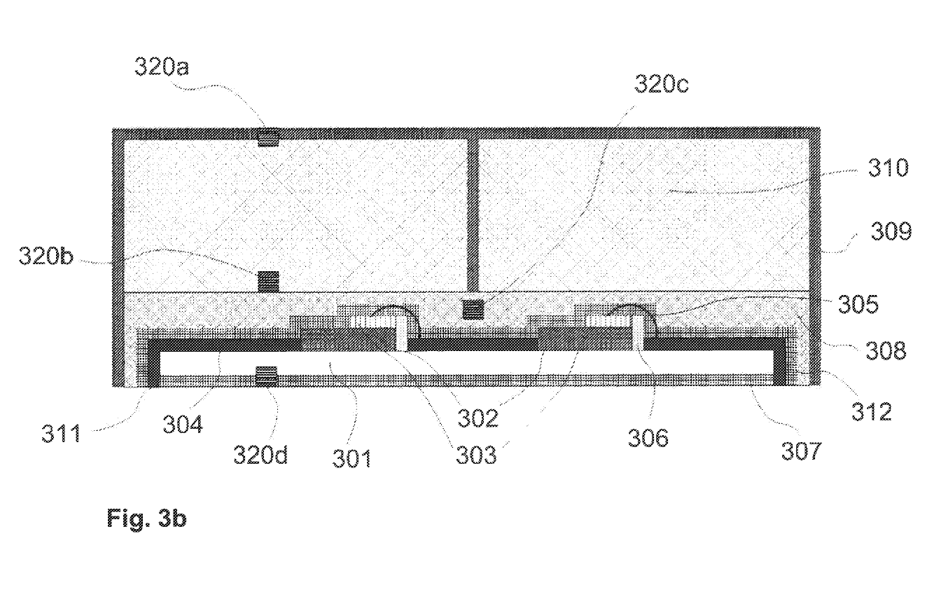

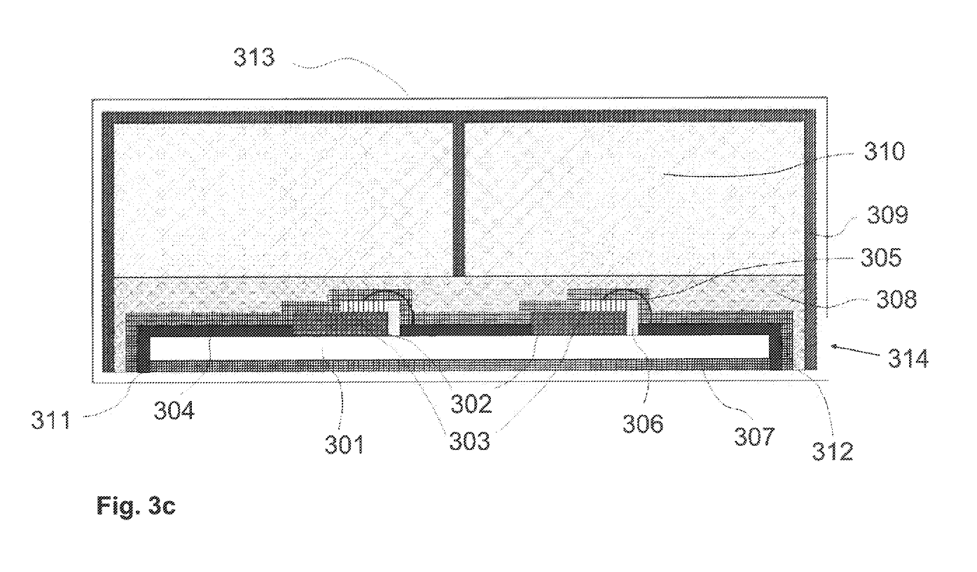

FIG. 3a depicts a schematic of an exemplary phototherapy gel pack, according to one embodiment of the disclosure. The gel pack is advantageously composed of layered parts, e.g., for less complex manufacturing. The layered parts may be bonded or sealed (e.g., bonded at the surfaces of the layered parts or sealed at the seams of the layered parts) to provide a waterproof structure of the gel pack. A gel pack may comprise at least one light source, which emits light through a gel in the gel pack and onto a target treatment area.

In some embodiments, the gel pack is formed by sealing a first layer 309 and a second layer 307 together in a waterproof manner to form the outer surface or covering of the gel pack, to prevent a gel 310, provided in between the first layer and the second layer, from leaking and protect the flexible circuit from outside water or moisture. The gel may be held by the first layer 309, between the first layer 309 and the second layer 307. General advantages relating to the provision of the gel are described at least in relation to FIG. 1.

The first layer may hold the gel in a plurality of compartments 330a and 330b (or more than two compartments), each compartment filled with the gel 310 to ensure a substantially even/equal distribution of the gel throughout the gel pack. The plurality of compartments may be advantageous to ensure a minimum distance (e.g., at least 5 mm) between the at least one light source and the target treatment area. The light emitted may be provided to the target treatment area more evenly, providing e.g., a more equal illumination profile. For light sources that produce heat, the even distribution of gel between the light sources and the target treatment area ensures a more evenly distribution of heat over the target treatment area, and in some embodiments, keeps the heated light sources far enough from the target treatment area to avoid burns.

A flexible circuit may be supported by the second layer 307. A flexible circuit having at least one light source, for instance the flexible circuit seen in FIG. 2, may be embedded in a gel pack. Because the flexible circuit may be embedded in the gel pack between waterproof layers, the gel pack and the flexible circuit together are waterproof. The flexible circuit may be embedded between the first layer 309 and the second layer 307. The flexible circuit has a layer of non-conductive substrate 301, which supports conductive elements thereon. One or more LEDs 303 may be mounted on thin plates of a conductive material 302 (or substrate). Said conductive material may be attached to one or more tracks 304 such that an array of LEDs may be formed. A track may include a conductive material, and connects the LEDs electrically in the LED array. In particular, a track may connect the thin plate of conductive material via a conductive wire 305 (or line). In some embodiments, an insulating material 306 (or substrate) may be provided next to the LED to electrically isolate the track coming from one LED from electrically contacting the thin conductive plate of a following LED to ensure a directed flow of current.

A gel pack may include at least two flexible and waterproof layers, e.g., sealed to form a flexible and waterproof covering of the gel pack. One of the layers may support said flexible circuit. Said one of the layers may support the side of the flexible circuit not having the at least one light source (“back side”). The side of the flexible circuit having the at least one light source (“front side”) may face towards a gel 310 that is held by the gel pack. The light emitted by the at least one light source may then travel through the gel, which is substantially transparent or translucent. During therapy, the gel is advantageously closer to the target treatment area than the flexible circuit to provide more efficient delivery of thermal therapy.

In one embodiment, at least one of the waterproof and flexible layer includes a second layer 307 provided on the back side of the flexible circuit. The layer 307 may include a thin, waterproof film. The film may be deposited on the flexible circuit having the at least one light source by, e.g., chemical vapor deposition (CVD) process, dip coating techniques, physical vapor deposition (PVD) process, or other suitable techniques. For instance, the water proof film may include polyimide, polymethylmethacyclate and/or any other suitable materials. Preferably, the film provides a smooth surface that is easy to clean or wipe with a cleansing solution, e.g., ethanol-alcohol solution. Further, the film provides protection for the flexible circuit to decrease the chance of tearing and/or puncture of the flexible circuit. The thin water protective film may be a relatively inexpensive and simple process for mass production. The film effectively seals the flexible circuit and/or embeds the flexible circuit in the gel pack, thereby protecting the flexible circuit from water or moisture. The flexible circuit may also be easily washable or cleaned due to the waterproof film. Furthermore, the film, as an added layer to the flexible circuit (which may be fragile or easily tearable) may improve the durability by providing added thickness and support to the flexible circuit. In some cases, the film as an added layer to support the flexible circuit provides better mechanical stability, e.g., as compared to a flexible circuit that is free to move or flow around the gel in a gel pack.

Alternatively or additionally, the second layer 307 may include a flexible waterproof sheet-like material and the flexible circuit is deposited thereon. The flexible waterproof sheet-like material is advantageously easy for a user to wash and/or clean. The flexible circuit may be adhered to the film by an adhesive, or other suitable bond or adhesive, between the nonconductive substrate 301 and the waterproof sheet-like material. The nonconductive substrate may itself be an adhesive that adheres, e.g., the conductive elements, to the flexible waterproof sheet-like material.

In some embodiments, the second layer 307 is colored to provide a visually appealing or decorative effect of the gel pack. In some embodiments, the layer 307 is opaque, such that the light emitted from the LEDs cannot pass through the opaque layer and into the eyes of the user (or other areas undesirable for the particular light therapy). In certain embodiments, the second layer 307 is translucent and/or substantially transparent, such that the user may appreciate the ability to see the flexible circuit.

Optionally, the front side of the flexible circuit may be provided with a flexible, waterproof film 312. The film may be transparent such that the light emitted from the LEDs on the front side may pass through. In some embodiments, the film may be made of a material or structures to filter the light emitted from the LEDs to achieve certain frequencies of light or patterns of light. The film advantageously protects the conductive elements from moisture or water or other interference from gel 310.

In some embodiments, a side of the flexible circuit having the at least one light source may be sealed by a sealing material 308, e.g., silicone gel, to provide (further) structural or chemical protection for the flexible circuit. The sealing material 308 may be applied before or after the film 312. Either the sealing material 308 or the film 312 is disposed against the gel held by layer 309. By providing the sealing material 308 and/or film 312 that holds the gel 310 and embeds the flexible circuit within the gel pack between the first layer 309 and the second layer 307, the layered (or sandwiched) structure may advantageously provides a gel pack that is waterproof. The sealing material and/or film may enable the flexible circuit to adhere and seal itself to the gel and the first layer, providing a watertight design. As such, users may place the gel pack (with the flexible circuit) into warm/hot water bath to heat up the gel. The gel pack may then release the stored heat during light therapy to provide a synergistic effect of light therapy and heat therapy. In some embodiments, the seams of the layered structure is additionally or alternatively sealed using suitable seals such as woven seams, heated seals, adhesives, etc.

Although not shown, it is appreciated that other layers may be deposited or provided to the first layer 309 and the second layer 307 (e.g., the thin, waterprotective film on the flexible circuit). For instance, a layer comprising at least one of decals, color, lettering, logos, instructions for use, warnings, etc. may be provided onto the waterproof film. In some embodiments, a gel and a further layer may be provided to the film 307, such that a gel pack is provided on the other side of the flexible circuit.

The gel pack may further include attachment parts or is removably provided with attachment parts such as hook-and-loop closure parts, buckles, clips, ties, etc. to allow the user to wear and/or strap the gel pack onto the user over the target treatment area. The attachment parts may be waterproof if the parts are not removable from the gel pack. The attachment parts may be disposable to enable easier reuse and replacement of the attachment parts of the wearable gel pack.

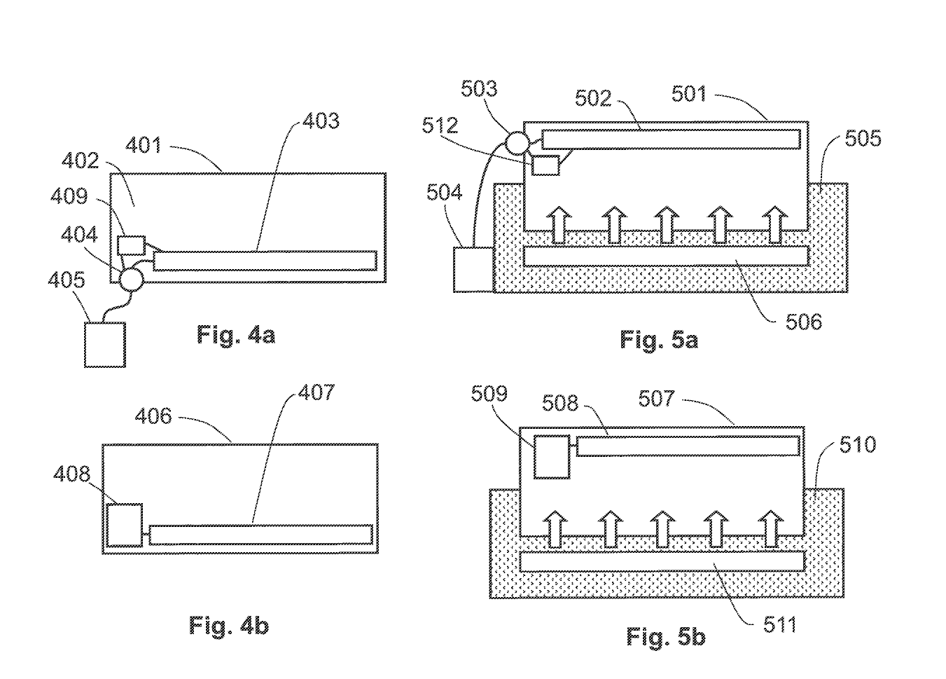

One or more tracks 304 may be electrically coupled and/or connected to a power source. Said power source (not shown) may be at least partially within the gel pack (built-in), or said power source may be outside the gel pack, electrically coupled to the one or more tracks through a waterproof connector 311. The power source may be removably coupled to the one or more tracks, and said waterproof connector is waterproof when the power source is connected or has been disconnected from the waterproof connector. Various embodiments relating to the power source are discussed in relation to FIGS. 4a, 4b, 5a, and 5b.

In some embodiments, the gel 310 provides thermal management, by absorbing the heat that may be produced by the at least one light sources of the flexible circuit. One or several thermistors 320 (see FIG. 3b) may be provided with the flexible circuit, electrically coupled to the light sources. The thermistor may measure the temperature of the gel, the light sources, the first layer, and/or other suitable place of the gel pack, such that the thermistor may turn off or reduce the light emitted by the at least one light source if a particular or threshold temperature is reached. A thermistor may be used to protect the target treatment area from overheating and/or over treatment. The thermistor may act as a timer to control the duration of treatment. The duration of the treatment may be determined by the thermal capacity of the gel and the flexible circuit and/or components thereon may be controlled and/or switched off once the gel has reached a certain temperature.

Depending on the application, the gel may be provided at different thicknesses. In an embodiment where higher flexibility is preferred, the gel may have a smaller thickness. A gel that has a smaller thickness may change temperature too quickly due to the small mass. For instance, if heat-producing light sources are used, or if an electric heating element is provided, then the gel thickness should be determined based on the amount of heat that is advantageously absorbed by the gel to ensure proper thermal effects on the target treatment area and/or to avoid overheating of the target treatment area. A gel that has a smaller mass may also be less effective in cold treatment because the gel would warm up too quickly. In contrast, if flexibility is less preferred, the gel may have a larger thickness, such that the gel pack may stay cold/hot longer or is able to absorb/buffer more heat from heat-producing light sources and/or electric heating elements. The thickness of the gel may range between approximately 0.4 centimeter and approximately 2 centimeter, wherein said thickness is substantially the distance between the target treatment area and the at least one light source.

FIG. 3c depicts a schematic of another exemplary phototherapy gel pack, according to one embodiment of the disclosure. For improved comfort for the target treatment area, an optional covering 313 may be used to hold the waterproof gel pack. An illustrative opening 314 is shown. The opening allows a user to insert the gel pack into the optional covering. The opening may further include hook-and-loop closure part or other suitable closure parts to keep the gel pack in the optional covering.

Waterproof materials such as soft plastic-like materials tend to stick to the target treatment area and/or adhere to body hair or skin when there is sweat or moisture induced by the hot/cold treatment. The material may cause discomfort to the target treatment area, where the disadvantageous are more pronounced when the target treatment area already requires medical attention. Furthermore, in some cases, the hot or cold treatment may be too strong in the beginning of treatment for the gel pack to directly contact the target treatment area. An optional covering may provide an added barrier to protect the target treatment from the waterproof outer surface(s)/material(s) of the gel pack strong hot or cold treatment.

In one embodiment, at least part of the optional covering may be made of a soft, cushioned, textile and/or woven material to provide improved comfort, while allowing at least some of the light emitted by the at least one light source on the flexible circuit to pass through the optional covering and onto the target treatment area. In some embodiments, the optional covering may be made with holes, cavities, slits, substantially transparent areas, translucent areas, filtering areas such that the light passing through may be configured for a particular therapy.

The optional covering may be relatively cheap such that the covering may be disposable. The disposability of the optional covering provides added sanitary benefits to allow the gel pack to be easily reused without substantial cleaning of the gel pack (reduce the frequency of cleaning).

The optional covering may provide aromas, scents, ointment, creams, gels, photosensitizer, or any suitable topical treatment/formula, (pre)deposited on the optional covering or other suitable treatments such that added therapeutic benefits may be provided during light therapy and the heat/cold therapy. For instance, ointments, creams, and/or gels may be absorbed more efficiently by the target treatment area during heat therapy while the target treatment area is being simultaneously treated with light therapy. Aromas and/or scents may be made stronger by the heat therapy to provide added aromatherapy benefits to the user. Creams, gels, and/or ointments, may be used to sooth the harshness of cold therapy at the target treatment area. The optional covering may provide a mildly adhesive material to allow the gel pack with the optional covering to adhere easily to the target treatment area to keep the gel pack in place.

The optional covering, may further include attachment parts or is removably provided with attachment parts such as hook-and-loop closure parts, buckles, clips, ties, etc. to allow the user to wear and/or strap the gel pack onto the user over the target treatment area.

FIG. 4a depicts a schematic of an exemplary phototherapy gel pack connectable to a power source, according to one embodiment of the disclosure. The gel pack 401 is provided with gel 402 (or any suitable gel-like material for storing heat or cold and/or slowly releasing heat or coldness stored therein). At least one electrical component 403 is embedded within the gel.

In one embodiment, at least one electrical components comprises a flexible circuit, e.g., such as the flexible circuit seen in FIG. 2, and said at least one electrical components is powered by an external power source 405, which may be electrically (and removably) coupled to the at least one electrical components through connection (or connector) 404. The connection and the external power source may be advantageously waterproof such that the assembly, including the external power source, may be, e.g., submersible in water. In particular, the connection may include an electrical connection part and a water shield which covers the electrical connection part when the external power source is engaged with the connection. The connection may be advantageously waterproof, even when the power source is disconnected. For instance, the connection may include a cap for sealing and/or protecting the connection from water when the power source is disconnected.

In some embodiments, the at least one electrical components may include electric heating elements (e.g., Joule heating), such that the gel may be heated using the power from power source 405. The power source may deliver power to heat the gel while the user is receiving light therapy, and/or the power source may deliver the power to heat the gel before use, depending on the implementation. In the later implementation, once the gel has been heated sufficiently, a sensor or thermistor (not shown) in the gel pack 401 may be used to automatically shut off and/or adjust the current from the power source to the heating elements. The sensor may also turn on or adjust an indicator (not shown) in the gel pack to indicate to the user that the gel pack has been sufficiently heated, e.g., so that the user may disconnect the power source at connector 404. The heat stored in the gel may then be slowly released to the target treatment area during light therapy.

The power source 405 may include a power outlet, a transformer, a power generator, a battery or other parts for storing/providing electrical energy. A suitable battery may be a one-time use battery or a rechargeable battery. The power source may optionally provide power to another power source (e.g. battery) 409 in the gel pack such that the power source 409 may be used to power the at least one electrical components 403 without connecting to power source 405 (e.g., to enable cordless use).

FIG. 4b depicts a schematic of another exemplary phototherapy gel pack having an internal power source, according to one embodiment of the disclosure. An internal power source 408 advantageously provides cordless use of the gel pack by internally providing power to at least one electrical component 407. Electrical components 407 having LEDs that may require relatively little power is advantageously powered by such an internal power source 408. A self contained gel pack including the power source and the electrical components improves portability of the gel pack, allowing the gel pack to be used even in places without good or any access to electricity. The entire gel pack, having the embedded internal power source, although usable for only a limited amount of time, may be entirely water waterproof to enable the gel pack to be heated in water and washed.

FIG. 5a depicts a schematic of an exemplary phototherapy gel pack connectable to a station, according to one embodiment of the disclosure. The station may enable an internal power source 512 to be charged and/or to pre-heat/cool the gel pack 501 before use. As seen in a similar gel pack of FIG. 4a, a gel pack 501 having at least one electrical component 502 embedded therein. The at least one electrical components may be electrically coupled to an external power source 504 through (waterproof) connector 503. In some embodiments, the gel pack includes an internal power source 512 if cordless use is desired, such that the external power source 504 may be disconnected (at connector 503) and power may still be delivered by the internal power source to the at least one electrical components.

In some embodiments, the gel pack may be heated and/or cooled before treatment to provide the hot/cold treatment such that the gel may release heat/cold to the target treatment area during light therapy. To enable convenient heating of the gel pack, a docking station 505 is provided to allow the gel pack to be heated and/or cooled by heating and/or cooling part 506. The external power source 504 may be provided with the charging station. At least one sensor (not shown) in the gel pack 501 and/or the docking station 505 may detect when the gel pack has been sufficiently heated and/or cooled. The data from the at least one sensor may be used to turn on/off/adjust an indicator to indicate to the user when the gel pack is ready (e.g., charged, hot or cold enough for use, etc.).

FIG. 5b depicts a schematic of another exemplary phototherapy gel pack having an internal power source, said gel pack connectable to a station, according to one embodiment of the disclosure. The gel pack 507 (similar to the gel pack seen in FIG. 4b) having an internal power source 509 for powering at least one electrical components 508, may also be heated/cooled by a heating/cooling element 511 of docking station 510. At least one sensor (not shown) in the gel pack 507 and/or the docking station 510 may detect when the gel pack has been sufficiently heated and/or cooled. The data from the at least one sensor may be used to turn on/off/adjust an indicator to indicate to the user when the gel pack is ready (e.g., charged, hot or cold enough for use, etc.).

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measured cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

It is to be understood that any feature described in relation to any one embodiment may be used alone, or in combination with other features described, and may also be used in combination with one or more features of any other of the embodiments, or any combination of any other of the embodiments. Moreover, the invention is not limited to the embodiments described above, which may be varied within the scope of the accompanying claims.

1. A phototherapy assembly, comprising:
a first waterproof and flexible layer applicable onto the target treatment area;
a second waterproof and flexible layer,
a gel in between the first layer and the second layer;
a flexible circuit in between the first layer and the second layer; and
at least one light source for providing a therapeutic effect to a target treatment area of a living being, the at least one light source coupled to said flexible circuit;
wherein:
the first layer and the second layer are sealed to provide a waterproof gel pack having the flexible circuit embedded in the waterproof gel pack; and
the at least one light source emits light through the gel and the first layer and 1 onto the target treatment area.

2. The phototherapy assembly of claim 1, wherein said second layer supports the flexible circuit.

3. The phototherapy assembly of claim 1, wherein the first layer, the gel, the second layer, and the flexible circuit are sealed together in a layered structure.

4. The phototherapy assembly of claim 1, further comprising a sealing material between the flexible circuit and the gel, said sealing material sealing the gel with the first layer and sealing the flexible circuit with the first layer and the gel.

5. The phototherapy assembly of claim 1, further comprising a seal that seals the seams of the first layer and the second layer to provide the waterproof gel pack.

6. The phototherapy assembly of claim 1, wherein said second waterproof and flexible layer comprises a first water protective film deposited on a first side of the flexible circuit.

7. The phototherapy assembly of claim 1, further comprising a second water protective film deposited on a second side of the flexible circuit, said second side of the flexible circuit having the at least one light source.

8. The phototherapy assembly of claim 1, wherein:
the flexible circuit further comprises at least one electrical heating part; and/or
the at least one light source comprises at least one heat producing light source.

9. The phototherapy assembly of claim 1, wherein the gel is held by the first layer in a plurality of compartments to provide substantially even distribution of the gel over the flexible circuit.

10. The phototherapy assembly of claim 1, further comprising:
an internal power source embedded in the gel pack, and electrically coupled to the flexible circuit; and/or
a waterproof connector and an external power source connectable to the waterproof connector to power the flexible circuit.

11. The phototherapy assembly of claim 1, further comprising:
a topical formula deposited on the first layer, said topical formula applicable to the target treatment area.

12. A phototherapy system comprising a phototherapy assembly according to claim 1, and a docking station, said docking station comprising:
at least one heating and/or cooling element for heating the gel of the gel pack; and/or
a power source for charging at least one power storage part of the gel pack.

13. A phototherapy system comprising the phototherapy assembly of claim 1 and a topical formula applicable to the target treatment area.

14. A topical formula for use in phototherapy, said use in phototherapy comprising:
applying the phototherapy assembly of claim 1 onto a target treatment area of a living being, wherein the phototherapy assembly is used as a hot pack or a cold pack;
providing the topical formula between the phototherapy assembly and the target treatment area.

15. The method of claim 14, wherein:
the topical formula is thermally activated and/or optically activated; and/or
the uptake and/or permeation of the topical formula at the target treatment area is increased during use due to heat and/or occlusion of the phototherapy assembly at the target treatment area.

16. The method of claim 14, wherein the topical formula includes a photosensitizer.

17. A phototherapy assembly of claim 1 for use in therapy, said use in therapy comprising:
applying the phototherapy assembly onto a target treatment area of a living being, wherein the phototherapy assembly is used as a hot pack or a cold pack;
providing the topical formula between the phototherapy assembly and the target treatment area.

18. The method of claim 17, wherein:
the topical formula is thermally activated and/or optically activated; and/or
the uptake and/or permeation of the topical formula at the target treatment area is increased during use due to heat and/or occlusion of the phototherapy assembly at the target treatment area.

19. The method of claim 17, wherein the topical formula includes a photosensitizer.

20. A method of increasing the uptake and/or permeation of the topical formula at a target treatment area, said method comprising:
applying a phototherapy assembly onto the target treatment area of a living being, a phototherapy assembly according to claim 1, wherein the phototherapy assembly is used as a hot pack and/or a cold pack;
providing a top

21. The method of claim 20, wherein:
the topical formula is thermally activated and/or optically activated; and/or
the uptake and/or permeation of the topical formula at the target treatment area is increased during use due to heat and/or occlusion of the phototherapy assembly at the target treatment area.

22. The method of claim 20, wherein the topical formula includes a photosensitizer.

23. A method of emitting light onto a target treatment area, said method comprising:
applying a phototherapy assembly onto the target treatment area of a living being, a phototherapy assembly according to claim 1, wherein the phototherapy assembly is used as a hot pack and/or a cold pack;
providing a topical formula between the phototherapy assembly and the target treatment area.

24. The method of claim 23, wherein:
the topical formula is thermally activated and/or optically activated; and/or
the uptake and/or permeation of the topical formula at the target treatment area is increased during use due to heat and/or occlusion of the phototherapy assembly at the target treatment area.

25. The method of claim 23, wherein the topical formula includes a photosensitizer.
